(19) United States
(12) Patent Application Publication
(10) Pub. No.: US 2003/0165402 A1
(43) Pub. Date: Sep. 4, 2003

(54) Biocidal protection system

(76) Inventors: Alex Sava, Paddington (AU); Steven Kritzler, Cronulla (AU)

Correspondence Address: JENKENS & GILCHRIST, A PROFESSIONAL CORPORATION, 1100 LOUISIANA, SUITE 1800, HOUSTON, TX, 77002-5214, US

(21) Appl. No.: 10/257,090

(22) PCT Filed: Apr. 5, 2001
(86) PCT No.: PCT/AU01/00380

(30) Foreign Application Priority Data
Apr. 7, 2000 (AU) PQ 6790

(51) Int. Cl. A61L 9/00; B01D 11/04; B01D 11/02

(52) U.S. Cl. 422/28; 422/32; 422/34; 422/36; 422/37; 422/256; 422/261

(57) ABSTRACT

The invention relates to a shelf stable liquid disinfectant concentrate composition containing at least 1% by weight of a quat biocide (biocidally active quaternary ammonium compounds) and capable of dilution with 20 parts of water to 1 part of concentrate to produce a diluted solution, the diluted solution exhibiting a MIC after 24 hours in the presence of up to 2% tryptone (or the protein equivalent thereof) which is less than the MIC of a simple solution of the same concentration of the same quat biocide in water in the presence of the same concentration of the protein. The biocidal efficacy of the quat biocide may be protected by an “activity protector” selected from the group consisting of “enzyme stabilisers”, “enzyme stabilising systems”, micelle formation modifiers and inhibitors, and combinations thereof. The invention also relates to a disinfectant working solution prepared from the concentrate, and to a method of protecting a quat biocide from deactivation.

### FIELD OF THE INVENTION

This invention relates to a biocidal system utilising a quaternary biocide and to a method of disinfection utilising the system.

### BACKGROUND ART

Quaternary ammonium compounds are a well known class of biocides. Of these monomeric quaternary ammonium compounds are more powerful antimicrobials and less costly than more recently developed polymeric quaternary ammonium compounds. Although not all quaternary ammonium compounds have biocidal properties or have them to the same extent as each other, the correlation between biocidal properties and chemical structure has been the subject of extensive investigation reported in the literature. Those skilled in the art have no difficulty in distinguishing between those which are useful as biocides and those which are not useful for biocidal purposes. The abbreviation “quat. biocide” is herein used to refer to biocidally active quaternary ammonium compounds.

Quat. biocides such as, for example benzalkonium chlorides, have the major advantage that they are broad spectrum, low cost biocides useful for general disinfection. One of the main disadvantages exhibited by quat. biocides is that they are instantly deactivated in the presence of proteins and certain ions such as those found in hard water. While the precise mechanism for this deactivation is not well understood, theories relating generally to complexing/binding of the cationic site of the quat. biocide with anionic sites of the protein are widely accepted as being a cause of the deactivation. While polymeric quaternary compounds are known which do not suffer from these disadvantages to the same degree, they are significantly less effective and more costly in comparison with the monomeric quaternary biocides. Accordingly it would be advantageous to provide a system which would enhance the efficacy of quat. biocides, and especially of simple monomeric quat. biocides, in the presence of protein and other deactivators.

Because quat. biocides are so readily deactivated by protein they are generally unsuitable for use as disinfectants intended to be applied to surfaces which may have become contaminated with proteinaceous material—for example food preparation surfaces, food preparation machinery, kitchen walls, partitions and floors or the like, or working and other surfaces in hospitals, in dental or medical practices, or for disinfecting medical instruments, paraphernalia, or equipment. Moreover quat. biocides cannot be used biocidally in combination with enzymes (which are proteins) since they are deactivated by the protein and also because they immediately deactivate the enzyme.

A convenient measure of biocidal efficacy of a biocide is its Minimum Inhibitory Concentration (“MIC”). MIC is a measure of the minimum concentration of the biocide which succeeds in preventing bacterial growth in a culture during a specified time period, for example 24 hrs.

Another measure of biocidal efficacy is to count the kill rate for standard cultures treated with a predetermined concentration of biocide after a predetermined time. In Australia, biocides are graded according to tests of the latter kind specified by the TGA as Grade B “Hospital Dirty”, Grade A “Hospital Clean”, Grade C “household/commercial”. A copy of “The TGA Disinfectant Test” is annexed. The TGA tests are specified as TGO 54. Similar tests and classifications are applicable in other countries. Details of the MIC test are shown in “Bailey & Scott ‘Diagnostic Microbiology’, 8^{th} edition, 1990 at page 177. MIC tests referred to herein are conducted over 24 hrs.

A quat. biocide dissolved in water at a concentration which is sufficiently effective to be classified by the TGA as, for example, a Grade A disinfectant (“hospital grade, clean”) would be at least 10 times less effective in the presence of as little as 1% of a protein Put another way, approximately a ten fold increase in concentration of the active biocide would be required to achieve complete kill of bacteria in the presence of say 1% of a protein as could have been achieved by that biocide in the absence of the protein.

Straight chain and polymeric quaternary ammonium compounds have been proposed for use in laundry detergents not for their antimicrobial properties but for their static control properties; fabric softening benefit or as a cationic surfactant. Quaternary ammonium compounds used as softeners or as surfactants are either inherently not effective biocides, or their biocidal activity is deactivated by ions in the formulation or in the water, and in use in laundry detergents are substantially devoid of any biocidal effectiveness.

Liquid dishwashing compositions have employed quaternary ammonium compounds as cationic detergents in combination with non-ionic detergents to assist with oil/grease removal. Some contain small concentrations (e.g. 0.001%) of a quaternary ammonium salt to help to prevent any bacterial growths from developing in the detergent composition during lengthy storage in opened containers.

Disinfection of surfaces contaminated with proteins currently requires at least a 2-step procedure:

Step 1. Physico-mechanical removal of proteinous soil

Step 2. Disinfection of pre-cleaned surfaces

Often this should be followed by a third step:

Step 3. Rinsing off residual disinfectants. A major advantage of monomeric quat. biocides is that some of them do not require to be rinsed even from food-contacting surfaces when applied at low levels.

There remains a need for effective and economical surface disinfection in the presence of protein. It would be especially desirable to provide a single step procedure and composition for enzyme-enhanced cleaning and disinfecting protein soiled surfaces.

Any discussion of the prior art herein is not to be construed as indicative of the state of the common general knowledge in the field.

It is an object of the present invention to overcome or ameliorate at least one of the disadvantages of the prior art, or to provide a useful alternative.

It is an object of at least some of the preferred embodiments of the invention to provide a quat. biocide composition which remains effective for 24 hrs notwithstanding the presence of protein.

It is a further object of at least some of these preferred embodiments to provide a liquid concentrate quat. biocide composition which can be readily diluted with water to provide a working solution which remains biocidally effective for at least 24 hrs notwithstanding the presence of protein, and which in preferred forms of the invention is also effective for cleaning

It is a further object of the preferred embodiments to provide a method for substantially protecting a quat. biocide from deactivation by a protein, and compositions employing that method.

It is also an object of certain highly preferred embodiments of the present invention to provide a composition including a quat. biocide and which has a lower MIC in the presence of a substantial concentration of protein, than a simple solution of the same quat. biocide at the same concentration in water in the presence of the same concentration of the protein. By a “substantial concentration” of protein is meant a protein content equivalent to 2 wt. % of water soluble tryptone powder (OXOID product No. L42) by weight of the diluted solution. A protein content equivalent is defined as 16 g of water soluble protein per litre of water, that is to say, not less than 0.54 g per litre water of amino nitrogen as per analysis described in “Nitrogen Compounds. Methods for analysis of musts and wines”, pp 172-195; Ough, C. S.; Amerine, M. A. (1988), New York: Wiley-Interscience. It will be understood that improved effectiveness could be expected in the presence of less than 2 wt. % tryptone,(or its protein equivalent) and that, for some purposes, satisfactory effectiveness may be retained in the presence of levels greater than 2 wt. % of tryptone (or its protein equivalent).

### BRIEF DESCRIPTION OF THE INVENTION

According to a first aspect the invention provides a shelf stable liquid disinfectant concentrate composition including: at least 1% by weight of a quat biocide; and a protein; and wherein said composition is capable of dilution with 20 parts of water to 1 part of concentrate to produce a diluted solution, the diluted solution exhibiting a MIC after 24 hrs in the presence of up to 2% of tryptone (or the protein equivalent thereof) which is less than the MIC of a simple solution of the same concentration of the same quat biocide in water in the presence the same concentration of the protein.

In preferred embodiments of the invention the minimum amount of disinfectant in the diluted solution required to achieve complete kill of Pseudomonas aeroginosa when tested in accordance with the TGA 054 test in the presence of proteinaceous soil is reduced by at least 25% in comparison with a simple solution of the same disinfectant.

By “shelf stable” is meant that the composition retains at least 50% of its biocidal efficacy after 12 months storage in a sealed container at 18-25° C.

Preferred embodiments of the invention retain better than 98% biocidal efficacy under these conditions.

A concentrate according to the invention may be used at a working dilution in which it is diluted at least 20:1 (i.e. 20 parts of water to 1 part of concentrate) to provide a working solution. In some embodiments of the invention it may be diluted to a much greater extent e.g. 100:1 or 1000:1 or more. However a dilution of 20:1 is used herein for definitional purposes. A 20:1 working dilution is of greater biocidal efficacy than a control which consists of a corresponding simple solution of the same concentration of the same quat biocide in water. Furthermore a working dilution of the concentrate not only retains biocidal activity in the presence of substantial amounts (for example, 2% by wt. of the diluted solution) of protein, but also, surprisingly, exhibits noticeably greater efficacy than a control. Surprisingly, the achieved level of protection of quat. biocide is such that the shelf-stable composition may include proteins in the form of enzymes. In preferred embodiments of the invention the concentrate further includes one or more enzymes and nevertheless retains shelf stability in the concentrate and enzymatic activity in use when diluted as well as having improved biocidal efficacy in use.

Throughout this description MIC is as determined after 24 hrs. Preferably the MIC of a composition according to the invention is less than 75% of the MIC of the corresponding control composition and more preferably is less than 50%.

According to a second aspect the invention provides a shelf stable liquid disinfectant concentrate according to claim 1 suitable for use after dilution for disinfection in the presence of protein, said concentrate including: at least 1% by weight of quat biocide and; a protein; and an activity protector selected from the group consisting of “enzyme stabilisers”, “enzyme stabilising systems”, “micelle formation modifiers and inhibitors”, and combinations thereof.

Compositions according to the invention include an “activity protector” which prevents loss of biocidal efficacy of the quat. biocide. In preferred embodiments the “activity protector” comprises a boron compound, and more preferably a boron compound in combination with di-(propylene glycol) methyl ether (“DPM”) or analogues thereof. Boron compounds have previously been used to protect enzymes from being irreversibly denatured but have not previously been used to protect quat. biocidal activity in the presence of proteins. DPM is known to modify micelle formation. It is believed that the “activity protector” could equally utilise (1) one or more other compositions selected from those known to be effective in stabilising enzymes in liquid aqueous solutions, including enzyme stabilising compounds and systems (2) selected “micelle inhibitors”, and mixtures of (1) and (2). In highly preferred embodiments of the invention the “activity protector” is an “enzyme stabiliser” and more particularly is a suitable concentration of boron anions. Desirably these are solvated in a polyol and may be combined with enzyme stabilising synergists or adjutants. Preferred “micelle inhibitors” include species known to modify as well as to inhibit micelle formation and are selected from C1-C6 alkanols, C1-C6 diols, C2-C24 alkylene glycol ethers, alkylene glycol alkyl ethers, and mixtures thereof. A highly preferred “micelle inhibitor” is di-(propylene glycol) methyl ether (“DPM”).

It has been found that the addition of DPM to an enzyme stabiliser synergistically enhances the activity protection conferred on the quat. biocide without detrimental effect on the activity of an enzyme if present.

It is highly preferred that the quat. biocide is an aryl quat compound, preferably benzalkonium halide.

It is well known that enzymes may become denatured in storage, in the presence of other enzymes, and/or in the presence of antagonistic ions such as for example anionic surfactants, quaternary ammonium compounds and detergency “builders”. A number of enzyme stabilising systems have been developed and are well known in the enzyme formulation art. An example of an “enzyme stabilising system” is a boron compound (e.g. boric acid) which in the past has been used alone or with selected other adjuvants and or synergists (e.g. polyfunctional amino compounds, antioxidants, etc) to protect proteolytic and other enzymes in storage and in various products. It has been theorised that an enzyme stabilising system such as boron and calcium form intramolecular bonds which effectively cross-link or staple an active site of enzyme molecule so as to hold it in its active spatial configuration. Enzyme stabilisers have not hitherto been used to improve the biocidal activity of a quat. biocide. The present invention is based on the surprising discovery that at least some enzyme stabilising systems are effective in protecting the biocidal activity of high concentrations of quat. biocides, even in the presence of protein, and yet release the biocidal activity upon dilution.

In accordance with the present invention the ratio of “activity protector” e.g. boron to quat. biocide is preferably chosen to minimise the MIC of quat. biocide in the presence of a given level of protein. It will be understood that the present invention may be used in compositions which combine a quat. biocide with one or more enzymes. In a case in which an enzyme is present in addition to the quat. biocide and in which it is desired to retain the enzymatic activity of the enzyme as well as the biocidal activity of the quat, biocide then the quantity of “activity protector” required will need to be greater than that required to protect the enzyme and will need to be sufficient to stabilise the enzyme and protect the biocidal activity of the quat. biocide. Moreover if the composition is anticipated to come into contact with an external proteinaceous load additional to the enzyme then the “activity protector” concentration will need to be greater still

The inventor has discovered that boron surprisingly protects a quaternary biocide from deactivation by a protein in such a way and to such an extent that the MIC of the biocide is not increased in the presence of a protein. In preferred embodiments of the invention the MIC is dramatically reduced, for example, more than halved notwithstanding the presence of up to 2 wt. % based on the weight of working solution of protein. This allows the formulation of a wide range of new and useful compositions which remain effective as disinfectants or antibacterials in circumstances in which the prior art would be significantly less effective or not effective at all. The invention also enables storage-stable liquid biocidally effective compositions to be prepared with a lower concentration of quat. biocide and at much lower cost.

Without wishing to be bound by theory, the inventor speculates that polymeric borate ions associate with the cationic quat. biocide, thus protecting the quat from combining with proteins. When the formulation is diluted the polymeric ions become unstable and release the quat for disinfection. Alternatively, it may be that the biocidal activity of the quat. biocide significantly relates to denaturing proteins of cell membranes and that boron complexes with charged groups of non-living proteins and prevents wasting quat. on denaturing non-living proteins. In any case, as enzymes are structurally quite different from quat. biocides, and as the complete mechanism by which quat. biocides kill bacteria is also uncertain, it was not previously predictable that any enzyme stabiliser would be effective in maintaining the biocidal activity of a quat. biocide (an enzyme antagonist). The mechanism by which the activity of the quat biocide is maintained may be different from that whereby an enzyme is stabilised.

“Activity protectors” are discussed in more detail hereinafter

According to a third aspect the invention provides a composition according to the first aspect further including a nonionic surfactant

Preferably the nonionic surfactant is one or a combination of surfactants selected from the group consisting of ethoxylates or propoxylates and block copolymer of these.

According to a fourth aspect the invention provides a composition according to any one of the preceding aspects including one or more stabilised proteins and wherein the MIC of the biocide at a working dilution is not reduced by a further combination with up to 2 wt. % of protein equivalent by weight of diluted solution.

Compositions according to the invention may be used, for example, and without limitation as a surface spray or treatment for disinfection, aseptic cleansing formulations, for cleaning medical/dental instruments and equipment, for impregnation into cloths and sponges, etc. as well as in consumer products such as dishwasher detergents, household cleaners, shampoo's, disinfecting laundry compositions, and the like. A highly preferred embodiment of the invention, provides an economical effective cleaning and disinfecting composition which contains enzymes, is stable in storage in concentrated or dilute form and on dilution remains biocidal in the presence of protein.

According to a fifth aspect the invention provides a working solution of a disinfectant biocidally effective in the presence of a protein, said solution including at least 0.5% by weight of a quat biocide; and a protein and wherein said solution is capable of dilution with 20 parts of water to 1 part of concentrate to produce a diluted solution, the diluted solution exhibiting a MIC after 24 hrs in the presence of up to 2% of tryptone (or the protein equivalent thereof) which is less than the MIC of a simple solution of the same concentration of the same quat biocide in water in the presence the same concentration of the protein.

According to a sixth aspect the invention provides a working solution of a disinfectant biocidally effective in the presence of a protein including: at least 0.5% by weight of quat biocide;

a protein; and. an activity protector selected from the group consisting of. “enzyme stabilisers”, “enzyme stabilising systems”, “micelle formation modifiers and inhibitors”, and combinations thereof.

According to a seventh aspect the invention provides a method of protecting a quat biocide from deactivation including the steps of combining the quat biocide with an “activity protector” selected from the group consisting of enzyme stabilisers and micelle destabilises or combinations thereof.

According to other aspects, the invention provides a method of protecting or improving the efficacy of quat. biocides in the presence of a protein both in concentrated solutions and at working dilutions thereof and a method of cleaning protein soiled surfaces.

### BEST MODES OF PERFORMING THE INVENTION

The invention will now be more particularly described by way of example only with reference to various embodiments.

Example 1 gives the formulation of a composition which is a concentrate stable in storage but which in use is diluted with water from 200:1 to 1000:1 (parts/wt water to 1 part/wt concentrate). The diluted (200:1) solution is effective as a surface cleaning agent and leaves a disinfectant on the surface which prevents bacterial growth for at least 24 hrs after application. It is as effective if the surface is pretreated with, for example, 2 wt. % tryptone, or 2 wt. % yeast by weight of dilute solution.

### EXAMPLE 1

| | g/l |
|---|---|
| Benzyl dimethyl ammonium chloride, CAS 68424-85-1 | 150 |
| Sodium tetraborate decahydrate, CAS 12007-42-0 | 30 |
| Glycerin, CAS 56-81-5 | 25 |
| Terric GN9 (note 1) | 200 |
| Dipropylene Glycol Methyl Ether, CAS 34590-94-8 | 100 |
| Water balance to | 1000 |

note 1
Terric GN9 is ethoxylated nonylphenol available from ORICA and is a non-ionic surfactant

Preparation

The sodium tetraborate is dissolved/suspended in the glycerol at 80° C. The quaternary biocide and Terric GN9 (non-ionic detergent) are combined with the DPM and the pH adjusted with e.g. acetic acid to pH 7.2-7.3. The borate/glycerin solution is then combined with the quaternary biocide

Comparative Results

The formulation of example 1 and various compositions including subsets of the components of example 1 were prepared, diluted 20:1 and subjected to MIC tests as set out in table 1 part A. The tests were repeated with compositions further including various proteins as set out in parts B, C, and D In the following table 1, MIC was measured by the test method described in Bailey and Scott Diagnostic Microbiology, 8^{th} edition, 1990, p.177 using one of the most resistant to QUATs strains of Pseudomonas aeroginosa ATCC No. 15442. In table 1, “quat.” is an abbreviation for benzyl dimethyl ammonium chloride quaternary biocide”.

TABLE 1

| | | MIC, ppm | MIC, ppm |
|---|---|---|---|
| | composition | (no boron) | (with boron) |
| A. | quat. | 20* | 12 |
| | quat + DPM | 16 | 8 |
| | quat + GN9 | 25 | 8 |
| | quat + DPM + GN9 | 16 | <8 |
| B. | quat + 2 wt. % tryptone | 180* | 78 |
| | quat + DPM + 2 wt. % tryptone | 160 | 66 |
| | quat + GN9 + 2 wt. % tryptone | 162 | 56 |
| | quat + DPM + GN9 + 2 wt. % tryptone | 128 | 56 |
| C. | quat + 2 wt. % yeast | 240* | 108 |
| | quat + DPM + 2 wt. % yeasts | 200 | 74 |
| | quat + GN9 + 2 wt % yeasts | 200 | 86 |
| | quat + DPM + GN9 + 2 wt. % yeast | 200 | 52 |
| D. | quat + subtilisin (0.1% protease enzyme) | 50* | 25 |
| | quat + DPM + enzyme | 25 | 12 |
| | quat + GN9 + enzyme | 25 | 12 |
| | quat + dpm + GN9 + enzyme | 25 | 8 |

*indicates control (quat according to prior art, A alone, B, C, D with protein)

Table 1, part A compares the MIC of various quaternary ammonium biocidal compositions in the absence of boron and in the presence of boron (i.e. according to the invention) but in the absence of protein. In each case comparison may be made with a control—“quat” (no boron)

Table 1 part A shows that Terric GN9 deactivates the quat. biocide as would be expected. Unexpectedly, DPM enhances the activity of a quat. even in the presence of GN9, while in each case the combination with Boron according to the invention produces a marked improvement in biocidal efficacy in comparison with the combination lacking boron and with the control

Table 1 part B shows that in the presence of a protein (2 wt. % tryptone) and in the absence of boron the quaternary biocide is substantially deactivated. The degree of deactivation is reduced by DPM even in the presence of non ionic surfactant GN9. However, the addition of the boron anions at least halves the MIC in the presence of the protein in each case. Compositions with boron according to the invention have a much lower MIC than the control quat biocide with tryptone and no other additive. DPM unexpectedly enhances this effect

Table 1 part C shows corresponding results for a mixture of natural proteins in baking yeast, and table 1 part D shows the results for a third protein—proteolytic enzyme subtilisin. It is noteworthy that there is a further improvement in efficacy of the quaternary biocide (reduction in MIC) in each 25 case when DPM is combined with the boron (i.e. the combination of DPM with boron synergistically improves the activity protection of the boron) in comparison with compositions lacking boron or DPM. Moreover this synergism occurs notwithstanding the deactivation effect of GN9

A preferred embodiment of the invention is shown in example 2. The composition of example 2 is a concentrate intended for dilution 1 part/wt concentrate in 200 parts/wt water. The composition is intended for application as a pre-soak for surgical instruments

### EXAMPLE 2

| component | % w/w |
|---|---|
| A. | |
| Nonyl phenol ethoxylate (Terric GN9) | 3 |
| Di (propylene glycol) methyl ether | 5 |
| Perfume | .1 |
| Water | 15 |
| B. | |
| Sodium tetraborate decahydrate | 6 |
| Glycerol | 4 |
| water | 5 |
| C. | |
| Acetic acid to pH 7.2-7.3 | |
| D. | |
| Ethylene Glycol | 5 |
| 10% subtilisin (Alcalase 2.5 DL) | 3 |
| E. | |
| Benzalkonium Chloride 80% | 30 |
| Water | to 100 |

Premix Borax with hot water and glycerin, add to A, adjust pH, let the mixture cool down 30C and then slowly add premixed ingredients D. Then add water premixed with Benzalkonium Chloride

The Quat Biocide

The invention has been exemplified by reference to alkyl benzyl dimethyl ammonium chloride (also known as benzalkonium chloride) as the highly preferred quat. biocide. However those skilled in the art will recognise that other monomeric quaternary ammonium antimicrobial compounds may be used.

It is preferred that the quaternary ammonium antimicrobial compound is selected from the group having a general formula:

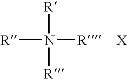

Wherein R′ R″ R′″ R″″ are alkyl radicals that may be the same or different, substituted or unsubstituted, branched or unbranched, and cyclic or acyclic. X is any anion but preferably a halogen, more preferable chlorine or bromine.

Highly preferred antimicrobial compounds are mono-long chain, tri-short chain, tetralkyl ammonium compounds, di-long-chain, di-short chain tetralkyl ammonium compounds and mixtures thereof, where by “long” chain is meant about C 6-C 30 alkyl, and by “short” chain is meant C 1-C 5 alkyl, preferably C1-C 3, or benzyl, or C 1-C 3 alkylbenzyl. Examples include monoalkyltrimethyl ammonium salts such as cetyltrimethyl ammonium bromide (CTAB), monoalkyldimethylbenzyl compounds or dialkylbenzyl compounds. Quat. biocides such as chlorhexadine gluconate may be employed.

The most highly preferred compounds for use in the invention have at least one benzyl radical which may be a substituted benzyl. Examples include C 8-C 22 dimethyl benzyl ammonium chloride, C 8-C 22 dimethyl ethyl benzyl ammonium chloride and di-C 6-C 20 alkyl dimethyl ammonium chloride The quaternary ammonium compound is incorporated for broad spectrum (gram positive and gram negative) antibacterial properties and should be present at least in an amount which would be effective for that purpose in the absence of protein or other deactivator. It is surprising that compositions according to the invention have excellent shelf stability both in concentrated and dilute form

Activity Protector

According to the invention the biocidal activity of the quaternary biocide is in use protected by an “activity protector” which is a composition (an ion, compound, or combination thereof) selected from the group of known “enzyme stabilising systems” including both reversible and irreversible enzyme inhibitors such as described in “Handbook of Enzyme Inhibitors”, Zoliner H., 2^{nd} ed. VCH 1993. The preferred activity protector is a boron compound or more preferably a mixture of a boron compound and a polyol The boron compound may for example be boric acid, boric oxide, borax, or sodium ortho-, meta-, or pyro-borate. In some formulations it may be desirable to use a perborate, such as sodium perborate to obtain a bleaching effect The most preferred boron source is sodium tetraborate. The protective effect of the boron compound may be enhanced by the presence of formate, or calcium ion, or by polyfunctional amino compounds such as di- or tri-ethanolamine. Other activity protection enhancers, or adjuvants, include anions such as phosphates, citrates, sulphates and sequestering agents such as used as water softeners such as EDTA.

Polyol

In systems which use boron to stabilise enzymes the addition of antioxidants is and/or polyfunctional amino compounds has been reported to produce a synergistic enzyme stabilising effect and the use of such enzyme stabiliser synergists in the present system is contemplated. The term “enzyme stabiliser systems” is used herein to denote combinations of stabilisers with enhancers, adjuvants and/or synergists and the like.

The polyol is preferably one containing from 2-6 hydroxyl groups and containing only C, H, and O atoms. Typical examples are ethylene glycol, propylene glycol 1,2 propanediol, butyleneglycol and most preferably glycerol. Other polyols such as mannitol, sorbitol, erythritol, glucose, fructose, lactose, etc may also be useful. The polyol is selected to solvate the boron and increase its ionic strength in the composition and will usually be present in an amount at least equal to the amount of boron compound.

Micelle Inhibitors

A water miscible solvent is desirably included to assist in solubilising the components and/or substances with which the composition comes into contact depending on its intended use and avoid or inhibit or modify micelle formation.

This acts synergistically as an “activity protector” as well as apparently in some instances enhancing biocidal activity in its own right.

Preferably a water miscible solvent is selected from C1-C 6 alkanol, C1-C 6 diols, C3-C 24 alkylene glycol ethers, alkylene glycol alky ethers and mixtures thereof. A highly preferred solvent is di (propylene glycol) methyl ether. Other known micelle antagonists include borates, lactates, citrates, tartrates.

Enzymes

The boron stabiliser is added in an amount required to prevent deactivation of the surfactant in the presence of protein. Surprisingly it has been found possible to include one or more enzymes in compositions according to the invention and to provide sufficient boron in the composition both to protect the quat. biocide from deactivation by the enzyme, and to protect the quat. biocide from deactivation by an additional protein (i.e. additional to the enzyme).and also to stabilise the enzymes against being denatured by the quat. It may be that a complex of the quat (e.g. with the protein) participates in reversibly protecting the enzyme. The enzymes may for example be proteolytic enzymes or selected from carbohydrases, esterases, hydrazes, amylases, proteases, catalases, lipases, amylases, cellulases, peroxidases, invertases, and the like together with mixtures thereof.

Surfactant

In preferred embodiments of the invention a surfactant is present. The surfactant is a non-ionic surfactant and it is highly preferred that it be selected from alkoxylated alcohols, alkoxylated phenol ethers. Other semipolar nonionics such as trialkyl amine oxides may also be useful. Examples of alkoxylated phenol ethers include octyl or nonyl phenol ether with varying degrees of alkoxylation. 6-10 moles of ethylene oxide per mole of phenol is preferred. The alkyl group can vary from C 6-C 16 The more highly preferred are low alkoxylated nonionics having 6-25 moles of ethylene oxide and/or propylene oxide per molecule.

The alkoxylated alcohols include ethoxylated and propoxylated C 6-C 16 alcohols with about 2-10 moles of ethylene oxide, or 1-10 and 1-10 moles of ethylene and propylene oxide per mole of alcohol respectively.

If amine oxides are used these may be mono-long chain, di-short chain, trialkylamine oxides and can be ethoxylated or propoxylated. an example is lauryl amine oxide, or cocoamidopropyldimethylamine oxide.

The quantity of surfactant is chosen so as to provide sufficient detergency for soil removal, and will typically be in the range of from 0.05% to 10% of the concentrate more preferably about 0.5% to 6% and most preferably from 2%-4%.

The fact that certain monomeric quat. biocides do not require to be rinsed even from food-contacting surfaces when applied at low levels provides an opportunity to formulate single-step cleaners/disinfectants, useful on food contacting surfaces soiled by proteinous soils.

The invention is herein described with particular reference to boron as the “activity protector”. It may be that not all enzyme stabiliser systems are effective as quat. biocide activity protectors, but those which are and are not effective can be determined by routine screening based upon the teaching hereof. The invention may-be embodied in many forms which will be apparent to those skilled in the arts of product formulation based upon the teaching herein contained.

Schedule 1

### The TGA Disinfectant Test

This test method has been reproduced wit the kind permission of the author and publisher from an original paper published in the “Australian Journal of Hospital Pharmacy”, Vol 8, No 4: 1978 (152-155)

1. Principle

The method, as applied to Hospital Grade Disinfectants or Sanitisers, is essentially that given by Kelsey & Maurer (1) for testing disinfectant performance. It is set out in a form suitable for attachment to a regulatory minimum standard for disinfectants and antiseptics. For wider application of the test refer to supplementary note A.

The disinfectant is tested at the dilution recommended by the manufacturer on the product label. The test consists of challenging the diluted disinfectant with bacterial inoculum, withdrawing a sample after a given time and culturing the sample in a suitable recovery medium. After this sampling, the mixture is again challenged by a second inoculum and after a second interval is again sampled for culturing. The sample is passed or failed according to the extent of growth shown in the two cultures sampled. The test may be performed with or without the addition of sterile yeast as an organic soil. (Options B and A respectively) or both according to the use-situations advocated on the label of the product under test.

TABLE 1
Selection of test parameters for classes of disinfectant
and antiseptic using the TGA Disinfectant Test.

| | | Test option | | |
|---|---|---|---|---|
| | | for re- | | |
| | | suspension | Number | |
| | Organisms | of | of | |
| Class of | used | centrifuged | chal- | Inoculum |
| product | in the test | organisms | lenges | density |
| Disinfectant - | Ps. aeruginosa | A (“clean” | 2 | 2 × 10⁸ − |
| hospital grade: | Pr. vulgaris | conditions) | | 2 × 10⁹ |
| Sanitiser | E. coli | B (“dirty” | | |
| | S. aureus | conditions”) | | |
| Disinfectant - | E. coli | C | 1 | 2 × 10⁸ − |
| household or | S. aureus | | | 2 × 10⁹ |
| commercial | | | | |
| grade | | | | |
| Antiseptic | Ps. aeruginosa | D | 1 | 1 × 10⁶− |
| (excluding | Pr. vulgaris | | | 1 × 10⁷ |
| those for intact | E. coli | | | |
| skin only) | S. aureus | | | |

For Household Grade disinfectants, the first two organisms listed and the second challenge are omitted, while Option C (nutrient broth) is selected as the choice of simulated soil. For antiseptics, the second challenge is again omitted, while Option D (serum) is selected as the choice of soil.

2. Media

All media must be contained in capped glass containers. Where media are stored, the containers must be sealed tightly or refrigerated.

2.1 Sterile Hard Water

2.1.1 Dissolve 0.304 g anhydrous calcium chloride and 0.065 g anhydrous magnesium chloride in glass-distilled water, and make up to one litre.

2.1.2 Dispense into glass containers and sterilize by autoclaving at 121°±1° C. for 15 minutes.

2.2 Yeast Suspension

2.2.1 Weigh 200 g of moist compressed baker's yeast. Cream by the gradual addition of sterile hard water using a heavy glass rod for stirring. Decant the creamed portion into a flask, add more water to any lumpy residue remaining and repeat the creaming and decantation until no residue remains and 500 ml of water has been used.

2.2.2 Shake the contents of the flask vigorously and strain through a 100-mesh sieve, breaking down any remaining lumps.

2.2.3 Add 500 ml sterile hard water, shake vigorously and adjust the pH to 6.9-7.1 with 1N Sodium hydroxide.

2.2.4 Transfer 50 ml, 100 ml or 200 ml of the yeast solution into screw-capped bottles.

2.2.5 Autoclave at 121°±1° C. for 15 minutes and allow the autoclave to cool without releasing pressure. Store cold but not freezing.

2.2.6 Dry two Petri dishes to constant weight. Into each, pipette 25 ml of sterilised yeast suspension, and dry to constant weight at 100° C. Calculate the average solids content of the suspension.

2.2.7 Before use, pipette 25 ml of the sterilised yeast suspension into a beaker. Determine the pH using the glass electrode, and determine the volume of 1N sodium hydroxide solution needed to adjust the pH to within the range 6.9 to 7.1.

2.2.8 Immediately before use, add to each bottle of sterilised yeast, a volume of sterile hard water and a volume of 1N sodium hydroxide calculated to adjust the concentration of dry yeast to 5.0% and the pH to within the range 6.9-7.1. Discard prepared yeast 3 months after preparation.

2.3 Medium for Growth of Test Organisms

2.3.1 Prepare a 10% w/v dextrose solution in distilled water, and sterilise by autoclaving at 121°±1° C. for 15 minutes. Cool to room temperature.

2.3.2 Prepare Wright and Mundy medium following the author's procedure (2) or from a commercial product of the same composition (Note B) and sterilise by autoclaving at 121°±1° C. for 15 minutes. Cool to room temperature.

2.3.3 To each litre of Wright and Mundy medium prepared in 2.3.2 add 10 ml sterile dextrose solution prepared in 2.3.1.

2.3.4 Aseptically dispense in either 10 ml or 15 ml amounts, as preferred.

2.3.5 This medium is referred to as Wright and Mundy dextrose medium.

2.4 Recovery Medium

2.4.1 Prepare nutrient broth as follows or from a commercial product of the same composition (Note B):—

Add the following to 970 ml of water and dissolve by heating.

| | | |
|---|---|---|
| Beef Extract Powder | 10 | g |
| Peptone | 10 | g |
| Sodium Chloride | 5 | g |

Adjust the pH to 8.0-8.4 using 1N Sodium Hydroxide.

Boil for 10 minutes and filter. Cool.

2.4.2 To each litre of nutrient broth solution prepared in 2.4.1 add 30 g polysorbate 80 (Note B).

2.4.3 Adjust pH to 7.2-7.4, using 1N Sodium hydroxide.

2.4.4 Autoclave at 121°±1° C. for 15 minutes, and immediately shake well to disperse the polysorbate 80.

2.4.5 Dispense aseptically in 10 ml amounts into sterile capped glass tubes.

3. Test Inoculum

3.1 Test Organisms

The following 4 organisms are to be used, except where prescribed.

| | |
|---|---|
| Pseudomonas aeruginosa | NCTC 6749 |
| Proteus vulgaris | NCTC 4635 |
| Escherichia coli | NCTC 8196 |
| Staphylococcus aureus | NCTC 4163 |

3.2 Preparation of Inoculum

3.2.1 Incubate the contents of an ampoule of freeze-dried culture overnight at 37°±1° C. in Wright and Mundy dextrose medium.

3.2.2 Inoculate the incubated culture onto nutrient agar slopes in McCartney bottles. Store for up to 3 months at 4°±1° C.

3.2.3 At a suitable period before the test is to be conducted, sub-culture from an agar slope into 10 ml or 15 ml quantities of Wright and Mundy dextrose medium. Incubate at 37°±1° C. for 24±2 hours.

3.2.4 Sub-culture from the medium in 3.2.3 into fresh medium, using an inoculating loop of 4 mm in diameter. Incubate at 37°±1° C. for 24±2 hours.

3.2.5 Repeat step 3.2.4 daily. For the test procedure use only those cultures which have been sub-cultured at least 5, and not more than 14 times.

3.2.6 Filter test cultures of P. aeruginoso and S. aureus through sterile Whatmans No. 4 filter paper.

3.2.7 Centrifuge all test cultures until cells are compact, and remove supernatant with a Pasteur pipette.

3.2.8 Resuspend test organisms in the original volume of liquid (i.e. 10 ml or 15 ml), and shake for 1 minute with a few sterile glass beads.

3.2.8.1 For Option A, resuspend in sterile hard water.

3.2.8.2 For Option B, resuspend in a mixture of 4 parts yeast suspension (prepared as in 2.2) to 6 parts sterile hard water.

3.2.8.3 For Option C, resuspend in nutrient broth (prepared as in 2.4.1 and 2.4.3 and sterilised by autoclaving).

3.2.8.4 For Option D, resuspend in sterile hard water; dilute twice 1÷9 in sterile hard water; then add 8 ml of the last dilution to 2 ml sheep serum previously inactivated at 56° C. for 20 mins. and sterilised by filtration.

3.3 Enumeration of Inoculum

Immediately before testing, sample the resuspended inoculum and enumerate using 10-fold dilutions in quarter-strength Ringer's solution and the pour-plate technique. The number subsequently counted must represent not less than 2×10⁸ or more than 2×10⁹ organisms per millilitre (or 1×10⁸-1×10⁷ using Option D) or the test is considered invalid. Retain tube containing 10⁻⁷ dilution for use in controls (7.3 and 7.4).

4. Disinfectant Dilutions

Quantitatively dilute a sample of the disinfectant to the specified extent, using sterile hard water as diluent. Use not less than 10 ml or 10 g of sample for the first dilution, and not less than 1 ml of any dilution to prepare subsequent dilutions. Make all dilutions in glass containers on the day of testing. The glass containers must be twice rinsed in glass-distilled water, and sterilised.

5. Temperature

Where air-conditioning does not maintain test solutions at 21°±1° C., hold the containers in which the test is to be carried out in a waterbath at this temperature.

6. Test Procedure

Perform the following test using each of the four test organisms (3.1) except where the Standard directs otherwise. It is not necessary to test with all organisms simultaneously.

6.1 Add 3 ml of diluted disinfectant to a capped glass container.

6.2 Start a timing device. Immediately inoculate disinfectant with 1 ml of culture (prepared in 3.2) and mix by swirling.

6.3 At 8 minutes, subculture one drop (0.02 ml±0.002 ml) into each of 5 tubes containing recovery broth. To ensure delivery of 0.02 ml into the first tube of recovery broth at exactly 8 minutes, it will be necessary to withdraw a suitable amount from the disinfectant test mix shortly beforehand. This must be immediately preceded by vortexing. Surplus sample must be returned to the test mix. (See Note D).

6.4 Except where prescribed, at 10 minutes, inoculate disinfectant with a further 1 ml of culture, and mix by vortexing.

6.5 Except where prescribed, at 18 minutes, proceed as in 6.3.

6.6 Mix the contents of all rubes of recovery broth by vortexing. Incubate at 37°±1° C. for 48±2 hours.

6.7 Examine for growth and record results.

6.8 For each test organism repeat steps 6.1-6.7 on each of 2 subsequent days, using a fresh disinfectant dilution and a freshly prepared bacterial suspension.

Controls

7.1 Recovery Broth Contamination

Incubate one uninoculated tube of recovery broth at 37°±1° C. for 48±2 hours and examine for growth. If growth occurs, the test is considered invalid due to contamination of the recovery broth.

7.2 Disinfectant Contamination

To 1 tube of recovery broth, add 0.02 ml of diluted disinfectant. Incubate at 37°±1° C. for 48±2 hours. If growth occurs, the test is considered invalid. Growth in 7.2 but not 7.1 indicates contamination of the disinfectant test solution.

7.3 Fertility Test

To 1 tube of recovery broth, add 1.0 ml of the 10⁻⁷ dilution retained in 3.3. Incubate at 37°±1° C. for 48±2 hours and examine for growth. If no growth occurs, the test is considered invalid.

7.4 Inactivator Efficacy

To 1 tube of recovery broth, add 0.02 ml of diluted disinfectant and 1.0 ml of the 10⁻⁷ dilution retained in 3.3. Incubate at 37°±1° C. for 48±2 hours, and examine for growth. If no growth occurs, the test is considered invalid. Growth in 7.3 but not in 7.4 indicates inadequate inactivation of the disinfectant.

Procedure in Case of Invalid Controls

When any control renders the test invalid, the test is to be repeated. Fresh recovery broth is to be used if growth occurred in control 7.1 or if no growth occurred in controls 7.3 or 7.4.

Should disinfectant contamination be indicated by control 7.2 on both occasions, the disinfectant is considered to fail the test. Should inadequate inactivation of the disinfectant be indicated by control 7.4 on both occasions, the test is considered invalid (Note C).

9. Results

The dilution test passes the test if there is no apparent growth in at least two out of the five recovery broths specified in 6.3 and no apparent growth in at least two of the five recovery broths specified in 6.5 on all three occasions, using all four organisms.

### 10. REFERENCES

(1) Kelsey, J. C. and Maurer Isobel, M. Pharmaceutical Journal (UK) 213: 528-530, (1974).

(2) Wright Eleanore, S. and Mundy, R. A. Journal of Bacteriology 80: 279-280, (1960).

11. Supplementary Notes

A. For investigational, developmental or comparative purposes, it will be useful to add a third challenge thus performing a true capacity test, and to test at dilutions above and below the prescribed dilution. In such cases, Kelsey & Maurer's recommendations regarding the timing and organisation of the test should be carefully consulted. Abbreviations of the test may be considered for the routine test of production batches.

B. Wright & Mundy medium is commercially available as “Bacto Synthetic Broth”, A.O.A.C. Code No. 0352 (Difco Ltd.). The nutrient broth to be used is available as “Nutrient Broth—No. 2” (Oxoid Ltd.).

C. Where inadequate inactivation is indicated, investigations should be conducted to find an effective inactivator. Refer Mackinnon, I.H.J. Hyg (London) 73: 189-195, (1974).

D. The Oxford P-7000 sampler system with disposable plastic tips is recommended for the withdrawal of samples for subculturing.

Schedule 2

Acceptable Common Names

| Descriptive Name | Common Names |
|---|---|
| Sterilant | Sterilant |
| Instrument Grade - | Instrument grade - High level disinfectant or |
| high level | High Level Instrument Disinfectant or |
| disinfectant | Instrument Disinfectant - high level or |
| | High Level Instrument Grade Disinfectant or |
| | High Level Disinfectant or |
| | Instrument Grade Disinfectant |
| Instrument Grade - | Instrument Grade - intermediate level |
| intermediate level | disinfectant or |
| disinfectant | Intermediate Level Instrument grade |
| | Disinfectant, or |
| | Intermediate Level Instrument Disinfectant or |
| | Intermediate Level Disinfectant |
| Instrument Grade - | Instrument Grade - low level disinfectant or |
| low level | Low Level Instrument Grade Disinfectant, or |
| disinfectant | Low Level Disinfectant, or |
| | Instrument Grade Disinfectant - low level |
| Hospital grade | Disinfectant - hospital grade |
| disinfectant | Hospital Grade Disinfectant |
| (see Surface spray | |
| below if primarily | |
| for use as a spray) | |
| Household/Commercial | Disinfectant - household grade, or |
| grade disinfectant | Disinfectant - commercial grade, or |
| (see Surface spray | Household Grade Disinfectant, or |
| below if primarily | Commercial Grade Disinfectant |
| for use as a spray) | |
| Surface spray | Surface spray disinfectant - hospital grade, or |
| disinfectant | Surface spray disinfectant - household grade, or |
| | Surface spray disinfectant - commercial grade |
| Antibacterial clothes | Antibacterial (together with a word or words |
| preparation | indicating the nature of the product) |
| Sanitary fluid | Sanitary fluid |
| Sanitary powder | Sanitary powder |
| Sanitiser | Sanitiser, or |
| | Sanitising Solution, or |
| | Antibacterial (together with a word or |
| | words indicating the nature of the product) |

The claims of the invention are as follows:

1. A shelf stable liquid disinfectant concentrate composition including:
at least 1% by weight of a quat biocide; and
a protein; and
wherein said composition is capable of dilution with 20 parts of water to 1 part of concentrate to produce a diluted solution, the diluted solution exhibiting a MIC after 24 hrs in the presence of up to 2% of tryptone (or the protein equivalent thereof) which is less than the MIC of a simple solution of the same concentration of the same quat biocide in water in the presence the same concentration of the protein.

2. A shelf stable liquid disinfectant concentrate according to claim 1 suitable for use after dilution for disinfection in the presence of protein, said concentrate including:
at least 1% by weight of quat biocide and;
a protein; and
an activity protector selected from the group consisting of “enzyme stabilisers”, “enzyme stabilising systems”, “micelle formation modifiers and inhibitors”, and combinations thereof.

3. A concentrate according to claim 1 or claim 2 wherein the protein is an enzyme.

4. A concentrate according to claim 3 wherein the enzyme is selected from one or more of carbohydrases, esterases, hydrazes, amylases, proteases, catalases, lipases, amylases, cellulases, peroxidases, invertases, and the like together with mixtures thereof.

5. A concentrate according to claim 2 capable of dilution with 20 parts of water to 1 part of concentrate to produce a diluted solution, the diluted solution exhibiting a MIC after 24 hrs in the presence of up to 2% of tryptone (or the protein equivalent thereof) which is less than the MIC of a simple solution of the same concentration of the same quat biocide in water in the presence the same concentration of the protein.

6. A concentrate according to any one of the preceding claims wherein the quat biocide is a monomeric quaternary ammonium antimicrobial agent.

7. A concentrate according to any one of the preceding claims wherein the biocidal efficacy of the quat biocide is protected by an activity protector selected from the group consisting of boron compounds, polyols, formates, calcium ions, polyfunctional amino compounds, phosphates, citrates, sulphates and sequestering agents.

8. A concentrate according to any one of the preceding claims including a micelle immiscible solvent.

9. A concentrate according to the preceding claim wherein the micelle immiscible solvent is selected from the group consisting of C1-C 6 alkanols, C1-C 6 diols, C3-C 24 alkylene glycol ethers, alkylene glycol alky ethers, borates, lactates, citrates, tartrates and mixtures thereof.

10. A liquid disinfectant concentrate according to any one of the preceding claims which retains at least 75% of its biocidal efficacy after 12 months storage in a sealed container at 18-25° C.

11. A liquid disinfectant concentrate composition according to any one of the preceding claims which retains at least 90% of its biocidal efficacy after 12 months storage in a sealed container at 18-25° C.

12. A concentrate according to any one of the preceding claims including at least 10% by weight of quat biocide.

13. A concentrate according to any one of the preceding claims including at least 25% by weight of quat biocide.

14. A concentrate according to any one of the preceding claims such that when diluted by 20 parts of water to 1 part of concentrate, the diluted solution exhibits a MIC after 24 hrs in the presence of up to 2% of tryptone (or the protein equivalent thereof) which is less than 50% of the MIC of a simple solution of the same concentration of the same quat biocide in water in the presence the same concentration of the protein.

15. A concentrate according to any one of the preceding claims such that when diluted by 20 parts of water to 1 part of concentrate, the diluted solution exhibits a MIC after 24 hrs in the presence of up to 2% of tryptone (or the protein equivalent thereof) which is less than 40% of the MIC of a simple solution of the same concentration of the same quat biocide in water in the presence the same concentration of the protein.

16. A concentrate according to any one of the preceding claims further including at least one non ionic surfactant.

17. A concentrate according to any one of the preceding claims wherein the quat biocide is a monomeric quaternary ammonium antimicrobial compound selected from the group having a general formula: 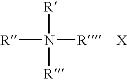
wherein R′ R″ R′″ R″″ are alkyl radicals that may be the same or different, substituted or unsubstituted, branched or unbranched, and cyclic or acyclic and X is any anion

18. A concentrate according to the preceding claim wherein X is chlorine or bromine.

19. A concentrate according to any one of the preceding claims wherein the quat biocide is selected from the group consisting of mono-long-alkyl chain, tri-short chain, tetralkyl ammonium compounds; di-long-chain, di-short chain tetralkyl ammonium compounds and mixtures thereof.

20. A concentrate according to the preceding claim wherein the quat biocide is selected from the group consisting of monoalkyltrimethyl ammonium salts, monoalkyldimethylbenzyl compounds, dialkylbenzyl compounds and quaternary gluconates

21. A concentrate according to any one of the preceding claims wherein the biocide is selected from the group consisting of C 8 to C 22 dimethyl benzyl ammonium chloride, C 8-C 22 dimethyl ethyl benzyl ammonium chloride and di-C 6-C 20 alkyl dimethyl ammonium chloride.

22. A concentrate according to any one of the preceding claims wherein the quat biocide is a benzyl dimethyl ammonium halide.

23. A concentrate according to the preceding claim wherein a stabiliser is selected from boric acid, boric oxide, borax, or sodium ortho-, meta-, or pyro-borate, perborates.

24. A concentrate according to the preceding claim wherein a stabiliser includes sodium tetraborate.

25. A concentrate according to any one of the preceding claims wherein the biocidal efficacy of the quat biocide is protected by a boron compound and further including a polyol having from 2 to 6 hydroxyl groups.

26. A concentrate according to the preceding claim wherein the polyol is selected from the group consisting of ethylene glycol, propylene glycol 1,2 propanediol, butyleneglycol and most preferably glycerol, mannitol, sorbitol, erythritol, glucose, fructose and lactose.

27. A concentrate according to claim 8 or 9 wherein the solvent includes di (propylene glycol) methyl ether (“DPM”).

28. A concentrate according to any one of the preceding claims including a surfactant selected from the group consisting of nonionic surfactants and semipolar nonionic surfactants.

29. A concentrate according to claim 28 wherein the surfactant is selected from the group including alkoxylated alcohols, alkoxylated phenol ethers, and trialkyl amine oxides.

30. A concentrate according to any one of the preceding claims including nonyl phenol ethoxylate.

31. A concentrate according to any one of the preceding claims after dilution by more than 200 parts of water to 1 part of concentrate.

32. A concentrate according to any one of the preceding claims after dilution by more than 1000 parts of water to 1 part of concentrate.

33. A working solution of a disinfectant biocidally effective in the presence of a protein, said solution including:
at least 0.5% by weight of a quat biocide; and
a protein
and wherein said solution is capable of dilution with 20 parts of water to 1 part of concentrate to produce a diluted solution, the diluted solution exhibiting a MIC after 24 hrs in the presence of up to 2% of tryptone (or the protein equivalent thereof) which is less than the MIC of a simple solution of the same concentration of the same quat biocide in water in the presence the same concentration of the protein.

34. A working solution of a disinfectant biocidally effective in the presence of a protein including:
at least 0.5% by weight of quat biocide;
a protein; and
an activity protector selected from the group consisting of “enzyme stabilisers”, “enzyme stabilising systems”, “micelle formation modifiers and inhibitors”, and combinations thereof.

35. A working solution according to claim 33 or claim 34 wherein the protein is an enzyme.

36. A working solution according to claim 35 wherein the enzyme is selected from one or more of carbohydrases, esterases, hydrazes, amylases, proteases, catalases, lipases, amylases, cellulases, peroxidases, invertases, and the like together with mixtures thereof.

37. A working solution according to any one of claims 33 to 36 wherein said protein is combined with the quat biocide prior to dilution to form the working solution.

38. A working solution according to any one of claims 33 to 36 wherein said protein is combined with the quat biocide on dilution to form the working solution.

39. A working solution according to any one of claims 33 to 36 wherein said protein is combined with the quat biocide after dilution to form the working solution.

40. A solution according to any one of claims 33 to 39 wherein the quat biocide is a monomeric quaternary ammonium antimicrobial agent.

41. A solution according to any one of claims 33 to 40 wherein the biocidal efficacy of the quat biocide is protected by one or more enzyme stabilisers and stabiliser enhancers selected from the group consisting of boron compounds, polyols, formates, calcium ions, polyfunctional amino compounds, phosphates, citrates, sulphates and sequestering agents.

42. A solution according to any one of claims 33 to 41 including a micelle immiscible solvent.

43. A solution according to any one of claims 33 to 42 wherein the micelle immiscible solvent is selected from the group consisting of C1-C 6 alkanols, C1-C 6 diols, C3-C 24 alkylene glycol ethers, alkylene glycol alky ethers, borates, lactates, citrates, tartrates and mixtures thereof.

44. A solution according to any one of claims 33 to 43 including at least 1.5% by weight of quat biocide

45. A solution according to any one of claims 33 to 44 including at least 2.5% by weight of quat biocide

46. A solution according to any one of claims 33 to 45 which exhibits a MIC after 24 hrs in the presence of up to 2% of tryptone (or the protein equivalent thereof) which is less than 50% of the MIC of a simple solution of the same concentration of the same quat biocide in water in the presence the same concentration of the protein.

47. A solution according to any one of claims 33 to 46 which exhibits a MIC after 24 hrs in the presence of up to 2% of tryptone (or the protein equivalent thereof) which is less than 40% of the MIC of a simple solution of the same concentration of the same quat biocide in water in the presence the same concentration of the protein.

48. A solution according to any one of claims 33 to 47 further including at least one non ionic surfactant.

49. A solution according to any one of claims 33 to 48 according to any one of the preceding claims wherein the quat biocide is a monomeric quaternary ammonium antimicrobial compound selected from the group having a general formula: 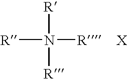
wherein R′ R″ R′″ R″″ are alkyl radicals that may be the same or different, substituted or unsubstituted, branched or unbranched, and cyclic or acyclic and X is any anion.

50. A solution according to any one of claims 33 to 49 wherein X is chlorine or bromine.

51. A solution according to any one of claims 33 to 50 wherein the quat biocide is selected from the group consisting of mono-long-alkyl chain, tri-short chain, tetralkyl ammonium compounds; di-long-chain, di-short chain tetralkyl ammonium compounds and mixtures thereof.

52. A solution according to any one of claims 33 to 51 wherein the quat biocide is selected from the group consisting of monoalkyltrimethyl ammonium salts, monoalkyldimethylbenzyl compounds, dialkylbenzyl compounds and Quaternary gluconates.

53. A solution according to any one of claims 33 to 52 wherein the biocide is selected from the group consisting of C 8 to C 22 dimethyl benzyl ammonium chloride, C 8-C 22 dimethyl ethyl benzyl ammonium chloride and di-C 6-C 20 alkyl dimethyl ammonium chloride.

54. A solution according to any one of claims 33 to 53 wherein the quat biocide is a benzyl dimethyl ammonium halide.

55. A solution according to any one of claims 33 to 54 wherein a stabiliser is selected from boric acid, boric oxide, borax, or sodium ortho-, meta-, or pyro-borate, perborates.

56. A solution according to any one of claims 33 to 55 wherein a stabiliser includes sodium tetraborate.

57. A solution according to any one of claims 33 to 56 wherein the biocidal efficacy of the quat biocide is protected by a boron compound and further including a polyol having from 2 to 6 hydroxyl groups.

58. A solution according to any one of claims 33 to 57 wherein the polyol is selected from the group consisting of ethylene glycol, propylene glycol 1,2-propanediol, butyleneglycol and most preferably glycerol, mannitol, sorbitol, erythritol, glucose, fructose and lactose.

59. A solution according to claim 42 or 43 wherein the solvent includes di (propylene glycol) methyl ether (“DPM”).

60. A solution according to any one of claims 33 to 59 including a surfactant selected from the group consisting of nonionic surfactants and semipolar nonionic surfactants

61. A solution according to claim 58 wherein the surfactant is selected from the group including alkoxylated alcohols, alkoxylated phenol ethers, and trialkyl amine oxides.

62. A solution according to any one of claims 33 to 61 including nonyl phenol ethoxylate.

63. A method of protecting a quat biocide from deactivation including the steps of combining the quat biocide with an “activity protector” selected from the group consisting of enzyme stabilisers and micelle destabilises or combinations thereof.

64. A method according to claim 64 wherein the activity protector is one or more substances selected from the group consisting of boron compounds, polyols, formates, calcium ions, polyfunctional amino compounds phosphates, citrates, sulphates and sequestering agents

65. A method according to claim 64 wherein the activity protector is one or more substances selected from boric acid, boric oxide, borax, or sodium ortho-, meta-, or pyro-borate, perborates.

66. A method according to claim 66 wherein the activity protector includes sodium tetraborate.

67. A method of disinfection of a surface including the step of diluting a concentrate according to any one of claims 1 to 33 with water and applying the diluted concentrate to the surface for an effective period.

68. A method of disinfection of a surface including the step of applying a solution according to anyone of claims 33 to 63 to the surface for an effective period.

69. A disinfectant substantially as herein described with reference to example 1 or example 2.
